(19) United States
(12) Patent Application Publication
(10) Pub. No.: US 2008/0021392 A1
(43) Pub. Date: Jan. 24, 2008

(54) MEDICAL FLUID ACCESS SITE WITH ANTISEPTIC INDICATOR

(76) Inventors: Kent L. Lurvey, Grayslake, IL (US); Dustin C. Cawthon, Crystal Lake, IL (US); Birendra K. Lal, Lake Zurich, IL (US); Vadim V. Krongauz, Bartlett, IL (US); Michael Tung-Kiung Ling, Vernon Hills, IL (US); Shmuel Sternberg, Palatine, IL (US); Rosa H. Yeh, Libertyville, IL (US); Robin G. Pauley, Lake Villa, IL (US)

Correspondence Address: BAXTER HEALTHCARE CORPORATION, ONE BAXTER PARKWAY, DF2-2E, DEERFIELD, IL, 60015, US

(21) Appl. No.: 11/458,816

(22) Filed: Jul. 20, 2006

(51) Int. Cl. A61M 5/00

(52) U.S. Cl. 604/111

(57) ABSTRACT

An access device including access site is disclosed for providing access to a medical fluid flow path for the introduction or withdrawal of medical fluids to and from the flow path. The access device includes an indicator for providing a visual indication when the access device has been exposed to an antiseptic agent.

### FIELD OF THE INVENTION

The present invention relates generally to medical fluid access devices for the addition or withdrawal of fluid to or from medical fluid flow systems.

### BACKGROUND OF THE INVENTION

Medical fluid access devices are commonly used in association with medical fluid containers and medical fluid flow systems that are connected to patients or other subjects undergoing diagnostic, therapeutic or other medical procedures. The access devices simplify the addition of fluids to or withdrawal of fluids from the container or the subject connected to the fluid flow system.

Within the medical field there are a wide variety of medical fluid flow systems, serving a variety of functions. One of the more common fluid flow systems is used for the intravenous administration of fluids, such as saline, antibiotics, or any number of other medically-related fluids, to a patient. These flow systems are commonly referred to as intravenous or “IV” fluid administration sets, and use plastic tubing to connect a phlebotomized subject to one or more medical fluid sources, such as intravenous solution or medicament containers.

Typically, such intravenous administration sets include one or more access devices providing access to the fluid flow path to allow fluid to be added to or withdrawn from the IV tubing. For example, an access device may allow for the introduction of medication, antibiotics, chemotherapeutic agents, or a myriad of other fluids to a previously established IV fluid flow system. Alternatively, the access device may be used for withdrawing fluid from the subject for testing or other purposes. The presence of one or more access devices in the IV tubing sets eliminates the need for phlebotomizing the subject repeatedly and allows for immediate administration of medication or other fluids directly into the subject.

Several different types of access devices are well known in the medical field. Although varying in the details of their construction, these devices usually include a housing that defines an access opening or site for the introduction or withdrawal of medical fluids through the housing, and a resilient gland that normally closes the access site. Beyond those common features, the design of access sites varies considerably. For example, the gland may be a solid rubber or latex septum or be made of other elastomeric material that is pierceable by a needle, so that fluid can be injected into or withdrawn from the access device. Alternatively, the gland may comprise a septum or the like with a preformed but normally closed aperture or slit that is adapted to receive a specially designed blunt cannula therethrough. Other types of access devices are designed for use with connecting apparatus employing standard male luers. Such an access device is commonly referred to as a “luer access device” or “luer-activated device,” or “LAD.” LADS of various forms or designs are illustrated in U.S. Pat. Nos. 6,682,509, 6,669,681, 6,039,302, 5,782,816, 5,730,418, 5,360,413, and 5,242,432, and U.S. Patent Application Publications Nos. 2003/0208165 and 2003/0141477, all of which are hereby incorporated by reference herein.

Access devices may also be fashioned as a part of a larger device or structure. For example, stop-cocks or the like used in medical fluid flow control may include access ports normally closed by septums or other elastomeric gland structures. Access devices or access sites may also be part of containers, such as vials or bags that have an opening closed by an elastomeric stopper or septum or other gland.

Before an access device is actually used to introduce or withdraw liquid from a container or a medical fluid flow system or other structure or system, good medical practice dictates that the gland and surrounding housing be contacted, usually by wiping or swabbing, with a disinfectant or sterilizing agent such as isopropyl alcohol or the like to reduce the potential for contaminating the fluid flow path and harming the patient. It will be appreciated that a medical fluid flow system, such as an IV administration set, provides a direct avenue into a patient's vascular system. Without proper aseptic techniques by the physician, nurse or other clinician, microbes, bacteria or other pathogens found on the surface of the access device could be introduced into the IV tubing and thus into the patient when fluid is introduced into or withdrawn through the access device. Accordingly, care is required to assure that proper aseptic techniques are used by the healthcare practitioner.

As described more fully below, the fluid access device of the present invention provides an important advance in the safe and efficient administration or withdrawal of medical fluids to or from a patient.

### SUMMARY OF THE INVENTION

In accordance with one aspect of the present invention, a medical fluid access device is provided, either alone or in combination with a medical fluid flow system, which includes a housing defining an access site for introduction or withdrawal of medical fluid through the housing and an indicator that provides a visual indication when the access site has been exposed to an antiseptic agent, such as by directly wiping or swabbing the access device pursuant to accepted aseptic practices.

The access device preferably also comprises a gland made of a resilient or other suitable elastomeric or other material and of any suitable design that normally closes the access site. For example, the gland may be solid so as to normally close the access site and be pierceable by a sharp cannula or needle to allow the introduction or withdrawal of medical fluid through the access device. Alternatively, the gland may have an aperture that is normally closed and is adapted to receive a blunt cannula therethrough for introduction or withdrawal of fluid. Still further the gland may be movable, such as upon pressure from a male luer or other flow member, between a normally closed position and an open position for introduction or withdrawal of medical fluid.

In one embodiment, the visual indication comprises a color change that is preferably but not necessarily of limited time duration. For example, the access device may have an original color, such as red, before exposure to an antiseptic agent. The original color may serve as a reminder to the clinician that the access site needs to be swabbed with antiseptic before accessing the site. When the access device is exposed to antiseptic agent, such as by direct swabbing by the clinician, the indicator will change color, such as to green to indicate that swabbing has taken place. The indicator may remain the changed color indefinitely or, more preferably, remain the changed color only for a limited time to indicate that antiseptic swabbing has taken place. After a limited time duration, the access device may eventually return to the original color, reminding the clinician to re-swab the access device before it is used again.

In accordance with another aspect of the present invention, the indicator which may comprise an agent disposed on the housing, on the gland, or both, may also serve as an indication of the degree of exposure or contact of the access device with an antiseptic agent. For example, the color change or the degree or amount of color change may serve as an indication to the clinician as to whether the access site has been actively swabbed, including using deliberate pressure, mechanical agitation, and duration of application in accordance with accepted aseptic practices. For example, the more intense color change may indicate a greater degree of contact with the antiseptic agent.

The access device may further include a fixed reference color that can be used by the clinician as a comparison to the color of the indicator. The reference color could be, for example, a ring of color disposed around the access site, such as in a “bulls eye” fashion, so that the clinician can compare the color change of the adjacent gland and/or housing to the fixed reference color after swabbing has taken place. This may help the clinician to determine when a complete color change has occurred, and consequently, indicate that active, deliberate swabbing has taken place.

In a related aspect of the present invention, the indicator may be located on a portion of the housing that generally circumscribes the access site. The indicator may be one or more of several types of indicators that operate on different principles. For example, the indicator may comprise a dye that changes color in the presence of a solvent in an antiseptic agent. Alternatively, the indicator may comprise a dye that changes color as a result of temperature change from the exposure to the antiseptic agent. Temperature change may occur during swabbing, for example, as a result of the cooler temperatures that occur as the antiseptic agent evaporates from the surface of the access device. In another embodiment, the indicator may be an agent impregnated in a portion of the housing, or the agent may be coated on a portion of the housing.

In another embodiment, the visual indication may comprise a change in transparency. For example, an indicator may be disposed on a portion of the housing, the gland, or both, and undergo a change in transparency upon contact with an antiseptic agent. The indicator may change, for example, from a substantially non-transparent state to at least a partially transparent state. More specifically, such an indicator may be comprised of a substantially non-transparent material, such as a layer of microporous membrane.

Further, the transparent material may be used in combination with a visual signal source that is obscured until the material becomes transparent upon contact with an antiseptic agent. When in a dry state, the non-transparent material may, for example, be white in color, and generally non-transparent so as to obscure the visual signal source. Upon contact with an antiseptic agent, the indicator may undergo a change in transparency to become at least partially transparent, allowing the visual signal source to become visible to the user. Such visual signal source may take a variety of forms, such as a particular color, a text message, an icon, or other visually communicative tool that provides a visual indication to the user that the access site has been exposed to antiseptic agent.

### BRIEF DESCRIPTION OF THE DRAWINGS

Turning now to a more detailed description of the various embodiments of the present invention illustrated in the attached drawings, of which:

FIG. 1 is a perspective view of a medical fluid flow system in flow communication with a phlebotomized human subject, that includes a fluid access device in accordance with the present invention for adding fluid to or withdrawing fluid from the medical fluid flow system.

FIG. 2 is a perspective view of one embodiment of a medical fluid access device embodying the present invention particularly well suited for attachment to a terminal end of fluid flow tubing, such as at the end of a catheter going into a patient.

FIGS. 3-5 are a sequence of perspective views of the access device of FIG. 2. FIG. 3 shows the access device prior to exposure to an antiseptic agent. FIG. 4 shows the access device being exposed to an antiseptic agent by swabbing the access site with an antiseptic cloth. FIG. 5 shows the access device providing a visual indication, such as by a change of color or transparency, that the access site has been exposed to an antiseptic agent.

FIG. 6 is a perspective view of another embodiment of the access device of the present invention as part of a medical fluid flow system, with fluid about to be added to or withdrawn from the flow system through the access device by a syringe with a needle.

FIG. 7 is a perspective view of another embodiment of a medical fluid access device of the general type shown in FIG. 2, and comprising a ring of a fixed reference color disposed on the access device for comparison by the user to evaluate a color change of the indicator when exposed to antiseptic agent.

FIG. 8 is a perspective view of the access device of FIG. 7 after the access device has undergone swabbing or other contact or exposure to an antiseptic agent and after the indicator has become substantially identical in color to the fixed reference color to indicate to the user that the access site has been disinfected in accordance with proper medical practice.

FIG. 9 is a side view of another embodiment of the access device of the present invention particularly suited for mounting in-line or mid-line in association with a fluid flow system.

FIG. 10 is a perspective view of the access device of FIG. 9 about to receive a male luer end of a syringe for introduction or withdrawal of medical fluid.

FIG. 11 is a perspective view of yet another embodiment of the fluid access device of the present invention located mid-line in a fluid flow path and adapted to receive a specially designed blunt cannula for introduction or withdrawal of medical fluid.

FIG. 12 is a graph generally showing a correlation between the time of swabbing the surface of an access device of the present invention with antiseptic agent, the reduction in the number of organisms on the surface of the access device, and the change in color intensity of a visual indicator that changes color upon exposure to an antiseptic agent.

### DETAILED DESCRIPTION OF THE INVENTION

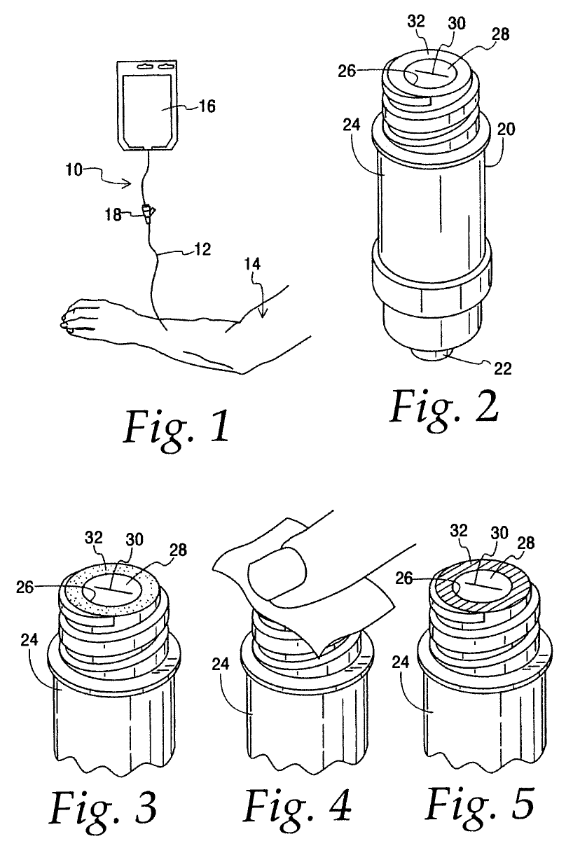

FIG. 1 generally illustrates a medical fluid flow system, generally at 10. The fluid flow system 10 is illustrated for exemplary purposes only as is an intravenous (IV) administration set. The present invention is not limited to IV flow systems and may be used in association with any apparatus, or container or flow system where fluid flow access is useful, such as but not limited to administering fluid to or withdrawing fluid from a subject (e.g. patient) or container (e.g. vial or bag).

As illustrated in FIG. 1, the set comprises a length of flexible plastic tubing 12, one end of which is connected via needle or other access means to a phlebotomized patient (i.e., subject) 14, and the other end is connected to a container 16 of medical fluid, such as intravenous solution or any other medically-related liquid. The tubing 12 of the IV administration set provides a fluid passageway allowing fluid communication between the donor 14 and the container medical fluid source 16.

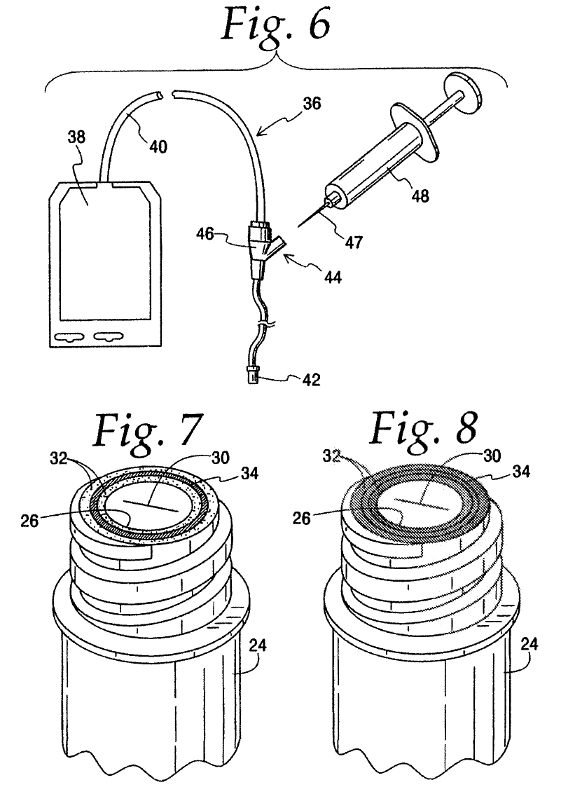

For introducing fluid into or withdrawing fluid from the flow path, the illustrated IV set 10 includes one or more access devices 18 in accordance with the present invention. The fluid access device 18 (or additional such devices) may be provided at any convenient location along the length of the tubing 12, and FIG. 1 illustrates a generally mid-line entry location. The access device 18 in FIG. 1 may take the form, for example, of the access devices shown in larger view in FIGS. 6, 9 and 11. Such access devices are commonly referred to as V-sites or Y-sites, in reference to their shape, and are typically but not exclusively used for mid-line entry points into fluid flow systems. Again, this is for purposes of illustration only. The fluid access device 18 of the present invention may be a permanent component of a fluid flow set or may be removably attached to a connector member or to tubing branch, without departing from the present invention—which is not limited to the general form or location of the access device. The fluid access device of the present invention may also be part of another structure such as a container, e.g., as a vial or bag, into which medical fluid is introduced or from which fluid is withdrawn.

Other shapes and forms of the access device may also be used. For example, FIG. 2 shows another access device 20 in accordance with the present invention that may be located at the terminal end of tubing 22, such as at the external end of an indwelling catheter inserted into a patient's blood vessel. Of course, the access device 20 could also be located at the end of any branch tubing associated with a fluid flow system 10. The illustrated fluid access device 20 has a generally elongated rigid or semi-rigid plastic housing 24 that includes a flow path (not shown) therethrough. The housing 24 material may be constructed of a semi-rigid or rigid medical grade material, such as polycarbonate material for ease of molding and bonding to common medical grade tubing, such as PVC tubing. The housing may be connected at its bottom or distal end to tubing 22 or to an intermediate connector as part of a fluid flow set. The top or proximal end of the housing 24 has a generally central circular opening or aperture 26 that defines an access site for introduction or withdrawal of medical fluid through the housing. As noted previously, such medical fluid may include, without limitation, IV solutions, blood and blood components, medications, chemotherapeutic agents, and various other fluids used for various diagnostic, therapeutic or other procedures.

In the illustrated access device, the aperture 26 is closed by a gland 28 that is preferably made of resilient/elastomeric material, such as rubber, silicone or latex. The gland normally closes and seals the aperture 26 when it is not being accessed. As used herein, “gland” is intended to have a broad and generic meaning directed to any member or members for normally closing or sealing the access site and which, in cooperation with an accessing member, allows for entry or access into the access site for introduction or withdrawal of medical fluid therethrough.

The gland 28 may be mounted or carried on or in the housing in a variety of ways that are known in the medical field. For example, as noted earlier, the gland may be a solid rubber, silicone or latex septum that spans the aperture and is pierceable by a needle, or the gland may include a pre-formed aperture or slit, which is adapted to receive or be opened by a blunt cannula, such as a male luer or specially designed cannula, for introducing or withdrawing fluid, or the gland may be moveable between the normally closed position and an open position, such as a “luer activated valve” or “luer access device” (LAD), for introduction or withdrawal of medical fluid. This is not an exclusive listing, but merely an indication of the wide variety of gland constructions that may be employed in an access site employing the present invention.

For purposes of illustration, FIGS. 2-5 show an access device in which the gland 28 has a preformed opening or slit 30 which is normally in a closed or sealed condition, but which can be forced open by penetration or compression by a blunt cannula or male luer. In this regard, the gland is preferably of resilient material so that it can be displaced or pierced in any suitable manner to allow access and return to a closed position sealing the aperture 26 and access site when the needle, cannula or luer is removed from the access site.

It will be appreciated that a fluid access device of the type described herein is, in normal usage, exposed to various contaminants or pathogens, such as airborne microorganisms, or by human contact by medical staff or patients. This may cause the surface of the access device to become contaminated with microbes such as bacteria or other harmful microorganisms. To reduce the risk of introducing such microorganisms or other pathogens into the subject, potentially resulting in infection, sickness, or even worse, good medical practice dictates that the clinician clean the access device with an antiseptic agent to kill or reduce the number of pathogens on the access site before administering fluid into or withdrawing fluid through the access site.

In accordance with the present invention, the access device 18 includes an indicator 32 that provides a visual indication when the access device has been exposed to an antiseptic agent. The indicator (or absence of a visual indication) serves as a deterrent to a clinician who might otherwise fail to observe proper aseptic techniques before introducing or withdrawing fluid through the access device.

As shown in FIGS. 2-5, the indicator 32 is located on the portion or edge of housing 24 that immediately surrounds the gland 28. Accordingly, when the medical personnel swab the surface of the gland, the indicator is also contacted by the antiseptic agent. In accordance with this aspect of the present invention, the indicator generates a visual indication to the user that such swabbing has occurred. The visual indication may be achieved in any suitable manner. It is preferable that the visual indication of exposure to the antiseptic agent be generated immediately upon disinfecting or shortly thereafter, for example, within several seconds.

As illustrated in FIGS. 2-5, the visual indication is a color change. The indicator has an original color before it has been exposed to an antiseptic agent, such as yellow, orange or red, and may change to another color, such as green or white or become transparent from opaque, upon exposure of the indicator to an antiseptic agent, such as by contacting the indicator by swabbing with an antiseptic agent. Where the original color of the indicator is red, orange or the like, it serves as a convenient and conspicuous warning or reminder to the clinician that the surface of the access device has not been properly treated with an antiseptic agent and that the access device should not be used until proper aseptic technique has been carried out. The changed color, such as green or white or other, indicates that the access site has been swabbed with disinfectant agent.

Referring to FIGS. 3-5, the clinician may treat the surface of the access device with an antiseptic agent by direct contact such as spraying, wetting, wiping or swabbing the gland and surrounding housing with a disinfectant or sterilizing agent. The antiseptic agent is typically contained in a cloth, tissue, cotton swab or the like, and by swabbing. Suitable sterilizing agents may include standard rubbing alcohol (isopropyl alcohol), or any other suitable antiseptic agents for killing bacteria or other pathogens.

A visual indication in the form of a color change may also serve as an indication to the clinician of the quality and thoroughness of the antiseptic treatment, such as proper swabbing surface coverage, proper swabbing force and/or pressure to the surface. For example, if aggressive antiseptic treating has taken place, the color change of the indicator may be of greater difference or intensity. More specifically, for example, if the indicator changes to green upon exposure to an antiseptic agent, the vividness or shade of the green color could indicate the extent of exposure. Dark or bright green could indicate that the access site has been aggressively treated with antiseptic agent or contacted with antiseptic agent over an extended period of time. A lighter or paler green might indicate a less aggressive treatment and signal the nurse or other clinician that further swabbing or wiping is necessary or would be useful.

It will be appreciated that the effectiveness of the antiseptic agent is only temporary, as the access device may become re-contaminated from exposure to the air or from human contact. As such, it is preferable that the visual indication, such as a color change, be temporary and of limited time duration, and that the access site return to its original or another color at some time after disinfecting—for example, several minutes, such as 3-10 or 5-10 minutes or such other time as may be desired after disinfecting. By reverting to the original condition or color, the indicator serves to remind the clinician to re-swab the access device with antiseptic agent before it is used again.

As seen in FIGS. 7 and 8, which are a variation on the embodiment of FIGS. 2-5, it may also be desirable to include a fixed reference indicator, such as a color, disposed on the gland or housing or both of the access device. As shown in detail in FIG. 7, the fixed reference may be a color in the form of a ring 34 or other similar marking of color on the access device. The reference color preferably is a color that is substantially similar to the color of the indicator after exposure to an antiseptic agent. In the illustrated access device, if the indicator turns green upon exposure to an antiseptic agent, the fixed reference color may be a green ring disposed near the access site, although the reference color could be at a location elsewhere on housing 24 significantly spaced from the access site. As shown in FIGS. 7 and 8, indicator 32 is a pair of rings flanking the reference ring 34 so that the colors may be easily compared and the indicator rings will be simultaneously contacted with antiseptic agent during swabbing. Swabbing is typically directed primarily at the gland and surrounding housing portions which may be contacted by the luer, cannula or needle for adding or withdrawing fluid through the access site. Thus, when the indicator is located on the housing, it is preferably located closely adjacent to the gland so as to contact the antiseptic agent to the same general degree as the gland.

With the above arrangement, if the indicator rings are, for example, red before the access device is swabbed with antiseptic agent, and the reference ring were green, the access device would have red, green and red rings around the gland prior to swabbing, thereby alerting the user to the need to disinfect the access site. After swabbing with an antiseptic agent, the red rings would turn green and the visual indication would appear as generally a single, wide green ring around the gland—until such time as the indicator rings 32 returned to their original red color or other non-green color.

The visual indicator of the present invention may also include a feature or aspect that facilitates visual indication to a clinicial who is color-blind or otherwise color-sight impaired. For example, if the visual indicator is a change of color, the change of color may have associated with it a pattern or arrangement that is visible to a color-impaired clinician. For example, the color change may be from a solid color to a color of having a pattern discernible to the impaired clinician, or vice versa. Alternatively, colors may be specially selected such that the color change employed by the indicator are detectable even by a person who is color-sight impaired.

The indicator of the present invention may be made in a number of different ways. In an optimal configuration, the indicator would actually be sensitive or reactive to presence of micro-organisms or other contaminants and would generate a visual indication when the surface of the gland and surrounding housing are substantially free of contaminants or microbes.

Alternatively, as indicated earlier, the indicator can be of a material that generates the visual indication in response to contact with an antiseptic agent, such as isopropyl alcohol. The indicator could, for example, include a solvatochromic dye that reacts to the polarity of the antiseptic solution. Such a dye could be coated on the access device or impregnated into the material of the access device. For a coating application to polycarbonate substrates, for example, the use of polyacrylic acid (hydrogel) with Nile red dye has yielded a blue to pink color shift in the presence of a polar solvent such as isopropanol. Alternatively, the dye could be incorporated into a film that is bonded to the access device.

Potential variants of this approach include the use of agents in the indicator that detect a pH shift or chemical change from the swab antiseptic agent. For stability, the active agent in the indicator, such as a dye or other agent, could be coated with another material or could be encapsulated in a hydrogel material or chemically cross-liked to other materials. Other variations may include cross-linked density hydrogels, placing the dye in an acrylic polymer such as HEMA, using thermal cure types of emulsion carriers for the dye, or employing lacquer carriers for the dye or employing substrates (such as silicone) to hold the dye in the substrate matrix. The dye could potentially also be suspended in a matrix of adhesive or epoxy or secured by two-shot molding of film to a polycarbonate substrate of the access device.

The indicator of the present invention could also include a liquid crystal material that changes color upon a temperature change from contact with the antiseptic agent. The liquid crystal material may be suspended between layers of film, such as polyester film, to capture the material. Such material may be present in the form of mirco-capsules. The film may then be attached to the access device housing, which may be of polycarbonate material, by cyanoacrylate adhesive or other bonding agent or method. Alternatively, the liquid crystal material may be suspended in an impregnated substrate or contained in a coating applied to the access device. An alternative to liquid crystal materials are thermochromic dyes, which change color due to a temperature change. Such dyes could be employed as described above. Such an indicator allows for quick color change which is also reversible back to the original color in minutes.

Yet a further variation of the indicator is the use of a pressure-sensitive material, such as liquid crystal microcapsules, which change color as a result of pressure exerted by the clinician during swabbing. Such microcapsules may exhibit a single color change and may not be reversible. Accordingly if reversibility of the color change is desired, other pressure sensitive materials or mechanisms may be employed.

In another embodiment, the visual indication may comprise a change in transparency. For example, an indicator may be disposed on a portion of the housing, the gland, or both, and undergo a change in transparency upon exposure to an antiseptic agent. For example, dry (air filled) microporous structures appear white to the observer even when the strands comprising the microporous structures are transparent. The reason this is so is the mismatch between the refractive index of the solid strand and the air. If the air is replaced by another fluid with a refractive index identical to or very close to that of the strand, the structure will appear transparent to the observer. It is desired that the fluid penetrate the structure if it is to wet all strands. One can use this phenomenon as a visual signal of good wetting by a known fluid. For example ePTFE and 70% IPA have almost identical refractive indices. Other membranes may include PTFE, PCTFE, PVDF, and ETFE membranes

Thus, when a microporous structure such as an ePTFE membrane which is white or opaque to the eye when dry, is wetted by swabbing with a 70% IPA, the membrane becomes transparent, and the underlying color or predetermined visual signal, such as printed message, will be visually discernible. If the user tries to swab with a fluid which does not provide effective swabbing, which by way of example may be water, the ePTFE membrane will not wet, and will remain white. This signaling procedure is purely physical in nature and does not rely on incorporated dyes or pigments and is not subject to extractables of any kind. In an example, the swabbing fluid may include 70% isopropyl alcohol. Chlorhexidine Gluconate with 70% IPA may also used be used as the swabbing agent as the 70% IPA matches the refractive index of such membranes.

Such a microporous structure may be carried or mounted on the gland or housing or both in any desired manner. If provided as a separate member, the microporous structure could be in the form of a membrane or other structure, with an adhesive backing that may be attached to the housing or gland using ultrasound, heat or other techniques.

The change in transparency may itself be a visual indication to the user, or the microporous structure may be used in combination with a visual signal source. When in a substantially non-transparent state, the microporous structure may serve to obscure the visual signal source, such as a source that is situated therebelow. Such a visual signal source may be, for example, a particular color, a text message, a bar code or other computer readable image, an icon, or other visual indicator that, when revealed, would provide an indication to the clinician that the access device has been exposed to antiseptic agent. Conversely, when the visual signal source is obscured by the non-transparent state of the indicator, the clinician will be alerted to the fact that the surface of the access device has not been treated with antiseptic agent. Accordingly, as the microporous structure dries, it returns to a non-transparent state, alerting the clinician to swab the access device before using it again. The reading of the visual signal source by a reader may then produce a signal which is fed into a medical information system or database to verify and produce a historical record of the swabbing before administration.

In a further embodiment the degree of transparency exhibited may be controlled such that the desired degree of transparency only occurs upon a desired level of swabbing. By way of example, a visual indicator such as a bar code may not be visible with a required degree of readability without the membrane being sufficiently wetted or swabbed, this required degree being imposed by the bar code reader. One method of achieving this control is to provide two or more layers of the membrane which require the desired swabbing before the needed transparency is achieved.

The accompanying drawings show other types of access sites in which the present invention also may be employed. FIG. 6, for example, illustrates a medical fluid flow system, generally at 36, having a medical fluid container 38 and flexible plastic tubing 40 extending between the container 38 and a terminal connector 42. The system 36 includes an access device 44 embodying the present invention and including a housing 46 and gland (not visible) with an indicator for indicating exposure to an antiseptic agent. The access device 44 is of the type that employs a solid septum, such as rubber, latex, or silicone, that is accessed by piercing with a needle, such as needle 47 attached to syringe 48. Examples of such an access site may be seen in U.S. Pat. Nos. 4,048,995 and 4,219,912, which are hereby incorporated by reference herein.

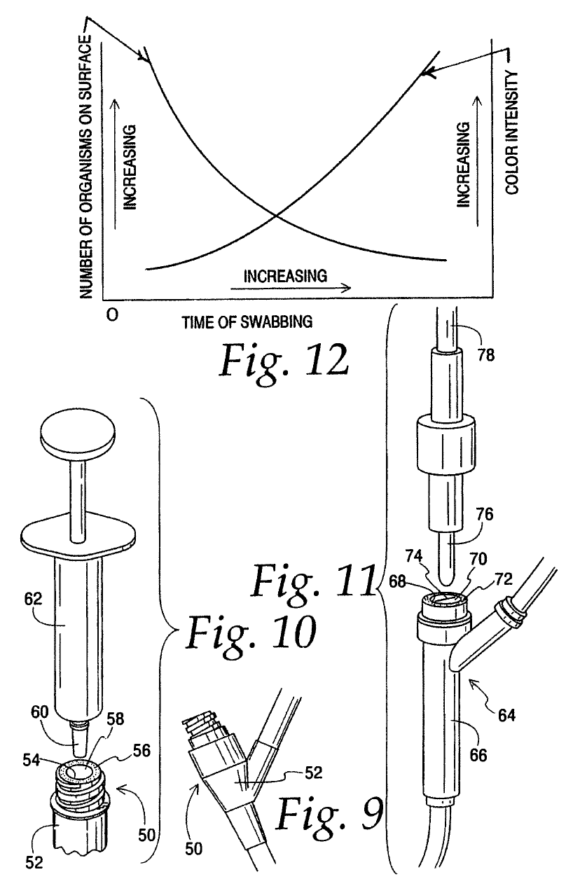

FIGS. 9 and 10 illustrate an access device 50 having a substantially rigid housing 52 having an opening or aperture 54 defining an access site and an indicator 56 for indicating exposure to an antiseptic agent. The device may further include a gland 58 for normally closing or sealing the aperture 54. There may be certain circumstances where the access site is not closed by a gland, but in most typical applications, a gland will be employed.

The access device of FIGS. 9 and 10 is a LAD-type device, for access by a male luer, and the gland 58 is depressed by contact with a standard male luer, such as the luer 60 illustrated on syringe 62, to open the access device to flow therethrough. Examples of this type of access site may be found in U.S. Pat. No. 6,682,509, where depression of a seal causes it to be pierced by an internal spike; U.S. Pat. No. 5,360,413, where depression of a piston accesses fluid passageways for flow through the access device; and U.S. Pat. No. 5,782,816, where compression of the valve element causes it to cant, permitting fluid flow through the device. All of the above patents are incorporated by reference herein and are intended to demonstrate that the present invention is not limited to the particular internal design or construction of a given access device, and that it has utility across the entire spectrum of access devices that are now or may later be employed in medical fluid access or flow systems.

In this regard, FIG. 11 illustrates a fluid access device 64 in accordance with another embodiment of the present invention and including a housing 66, an aperture or opening 68 in the housing defining an access site, a gland 70, normally closing and sealing the aperture, and a visual indicator 72. This access device has a preformed but normally closed opening or slit 74 for receiving a blunt cannula 76 of a connecting fluid flow system 78. The blunt cannula may be a specially designed cannula, such as described in U.S. Pat. No. 5,135,489, incorporated by reference herein, or may be a standard male luer or other member as illustrated, for example, in U.S. Pat. No. 6,669,681, also incorporated by reference herein.

It should be understood that the indicator may include a protective coating which is permeable to the swabbing fluid. Such a coating would not hinder the indication of proper swabbing from being verifiable but would act as a controller of proper IPA absorption relative to the quality of the swabbing conducted. In an example the coating would be a clear porous material.

Finally, it should be noted that the access device of the present invention does not need to be a separate device, and the access device housing may also be formed as part of another structure, such as in the form of the neck end of a medical fluid vial, a port on a stopcock or any other structure that defines an access opening into a container, a fluid flow system or other structure for the introduction or withdrawal of medical fluid therethrough. For example, the present invention may be particularly useful on medical vials. Such vials are commonly glass or plastic containers with an open top or neck defining an opening or access site that is sealed by an elastomeric gland such as a rubber or silicone stopper or septum. The visual indicator of the present invention may be employed on the gland, or on a surrounding portion of the vial or closure structure, or both, so as to indicate to the user when the stopper or septum has been properly swabbed with disinfectant before the stopper or septum is punctured or otherwise accessed for withdrawal of contents from or introduction of fluid into the vial.

While the present invention has been described in terms of certain preferred and alternative embodiments for purposes of illustration, it is not limited to the precise embodiments shown or to the particular features, shapes or sizes illustrated. A variety of changes may be made without departing from the present invention as defined by the appended claims.

1. A medical fluid access device with an exposure indicator comprising:
a housing defining an access site for introduction or withdrawal of medical fluid through the housing; and
an indicator that provides a visual indication when the access site has been exposed to an antiseptic agent.

2. The access device of claim 1 further comprising a resilient gland normally closing the access site.

3. The access device of claim 2 wherein the gland is selected from the group consisting of a gland that is pierceable by a needle for introduction or withdrawal of medical fluid, a gland that has a normally closed aperture adapted to receive a blunt cannula therethrough for introduction or withdrawal of medical fluid; and a gland that is moveable between the normally closed position and an open position for introduction or withdrawal of medical fluid.

4. The access device of claim 1 wherein the visual indication comprises a color change.

5. The access device of claim 4 wherein the color change is for a limited time duration.

6. The access device of claim 5 wherein the indicator has an original color before exposure to an antiseptic agent and returns substantially to the original color after the limited time duration.

7. The access device of claim 1 wherein the indicator indicates a degree of exposure to an antiseptic agent.

8. The access device of claim 2 wherein the indicator comprises an agent on the housing, the gland or both, which agent changes color upon contact with an antiseptic agent.

9. The access device of claim 8 wherein the degree of color change is indicative of the degree of contact with the antiseptic agent.

10. The access device of claim 4 further comprising a fixed reference color for comparison to the color of the indicator.

11. The access device of claim 1 wherein the indicator is located on a portion of housing that generally circumscribes the access site.

12. The access device of claim 1 wherein the indicator comprises a dye that changes color in the presence of a solvent in the antiseptic agent or in response to a temperature change.

13. The access device of claim 1 wherein the indicator comprises a liquid crystal agent that changes color as a result of a temperature change.

14. The access device of claim 1 wherein the indicator comprises an agent impregnated in at least a portion of the housing.

15. The access device of claim 1 wherein the indicator comprises an agent coated on at least a portion of the housing.

16. The access device of claim 1 wherein the indication comprises a change in transparency.

17. The access device of claim 1 wherein the visual indicator comprises a change from substantially non-transparent to at least partially transparent.

18. The access device of claim 16 wherein the indicator further includes a visual signal source that becomes visible upon a change in transparency when the indicator is contacted by an antiseptic agent.

19. The access device of claim 18 in which the visual signal source is selected from a group consisting of a color, a text message, and an icon.

20. The access device of claim 2 in which a portion of the housing or gland or both changes in transparency upon contact with an antiseptic agent.

21. The access device of claim 1 in which the indicator comprises a layer of microporous structure.

22. A medical fluid administration set comprising:
a fluid flow path for communicating between a fluid source and a patient;
the fluid flow path including an access device defining an access site for introducing fluid into or withdrawing fluid from the flow path; and
the access device comprising an indicator that provides a visual indication when the access site has been exposed to an antiseptic agent.

23. The medical fluid administration set of claim 22 further comprising a resilient gland normally closing the access site.

24. The medical fluid administration set of claim 23 wherein the gland is selected from the group consisting of a gland that is pierceable by a needle for introduction or withdrawal of medical fluid; a gland that has a normally closed aperture adapted to receive a blunt cannula therethrough for introduction or withdrawal of medical fluid; and a gland that is moveable between the normally closed position and an open position for introduction or withdrawal of medical fluid.

25. The medical fluid administration set of claim 22 wherein the visual indication comprises a color change.

26. The medical fluid administration set of claim 25 wherein the color change is for a limited time duration.

27. The medical fluid administration set of claim 26 wherein the indicator has original color before exposure to an antiseptic agent and returns substantially to the original color after the limited time duration.

28. The medical fluid administration set of claim 22 wherein the indicator indicates a degree of exposure to an antiseptic agent.

29. The medical fluid administration set of claim 23 wherein the indicator comprises an agent on the housing, the gland or both, which agent changes color upon contact with an antiseptic agent.

30. The medical fluid administration set of claim 29 wherein the degree of color change is indicative of the degree of contact with the antiseptic agent.

31. The medical fluid administration set of claim 25 further comprising a fixed reference color for comparison to the color of the indicator.

32. The medical fluid administration set of claim 22 wherein the indicator is located on a portion of housing that generally circumscribes the access site.

33. The medical fluid administration set of claim 22 wherein the indicator comprises a dye that changes color in the presence of a solvent in the antiseptic agent or in response to a temperature change.

34. The medical fluid administration set of claim 22 wherein the indicator comprises a liquid crystal agent that changes color as a result of a temperature change.

35. The medical fluid administration set of claim 22 wherein the indicator comprises an agent impregnated in at least a portion of the housing.

36. The medical fluid administration set of claim 22 wherein the indicator comprises an agent coated on at least a portion of the housing.

37. The medical fluid administration set of claim 22 wherein the indication comprises a change in transparency.

38. The medical fluid administration set of claim 22 wherein the visual indicator comprises a change from substantially non-transparent to at least partially transparent.

39. The medical fluid administration set of claim 37 wherein the indicator further includes a visual signal source that becomes visible upon a change in transparency when the indicator is contacted by an antiseptic agent.

40. The medical fluid administration set of claim 39 in which the visual signal source is selected from a group consisting of a color, a text message, and an icon.

41. The medical fluid administration set of claim 23 in which a portion of the housing or gland or both changes in transparency upon contact with an antiseptic agent.

42. The medical fluid administration set of claim 22 in which the indicator comprises a layer of microporous structure.
